(19) United States
(12) Patent Application Publication
(10) Pub. No.: US 2018/0129988 A1
(43) Pub. Date: May 10, 2018

(54) SYSTEM AND METHOD FOR CROSS-INSTITUTIONAL PROCESS OPTIMIZATION

(71) Applicant: Bernadette C. O'Connell de la Flor, Miami, FL (US)

(72) Inventor: Bernadette C. O'Connell de la Flor, Miami, FL (US)

(21) Appl. No.: 15/649,123

(22) Filed: Jul. 13, 2017

Related U.S. Application Data
(60) Provisional application No. 62/362,027, filed on Jul. 13, 2016.

(51) Int. Cl. G06Q 10/06; G16H 40/20; G16H 80/00

(52) U.S. Cl. CPC G06Q 10/0633; G06Q 10/0639; G06N 99/005; G16H 80/00; G16H 40/20

(57) ABSTRACT

There is presented a system and method designed to bridge workflow expertise from an enterprise with top or best-in-class outcomes to other enterprises so that the latter can improve existing processes to achieve learn to achieve the same or similar top outcomes. By providing real-time monitoring of operational performance compared against desired outcomes institutional issues can be mitigated in near-real time, and errors may be tracked and correlated to their respective sources to allow determination of proper courses of action. Further, push-type notifications may be sent to staff or management of the relevant institution to bring out-of-range operational issues to immediate attention, allowing timely mitigation and minimization of institutional impact.

### CROSS REFERENCE TO RELATED APPLICATIONS

This application claims full benefit of and priority to U.S. Provisional Patent Application No. 62/362,027 filed Jul. 13, 2016 titled, “SYSTEM AND METHOD FOR CROSS-INSTITUTIONAL PROCESS OPTIMIZATION,” the disclosure of which is fully incorporated herein by reference for all purposes.

### NOTICE OF INCLUDED COPYRIGHTED MATERIAL

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. All trademarks and service marks identified herein are owned by the applicant.

### FIELD AND BACKGROUND OF THE INVENTION

### Field of the Invention

The present disclosure generally relates to a system and method designed to bridge workflow expertise from an enterprise with top or best-in-class outcomes to other enterprises so that the latter can improve existing processes to achieve learn to achieve the same or similar top outcomes.

### Background of the Invention

Some enterprises have optimized workflows to achieve optimal outcomes. These workflows can be replicated. However, current attempts to transfer the knowledge to replicate these workflows have been inadequate and limited in scope and scale to enable subsequent enterprises to replicate the top outcomes.

Performance Based Training Assessment U.S. Pat. No. 7,181,413B2 is an example of training assessment efforts that fall short of affecting improvements and relies on trial and error to determine if developing certain skills sets in individuals will render a desired outcome in workflow.

Human Factor Management System of Nuclear Power Plant and Method CN 101847222 describes an example of a system and method that seeks to identify the key factors and classify the problem, management, and intelligent assessment system for facilities and people however falls short of what to do once the key factors have been identified and completed the above classification.

Another example in the art is shown in System and Method for Performing an Action on a Structure in Computer-Generated Data as set forth in U.S. Pat. No. 5,946,647. In this invention there is shown an analyzer that classifies without predicting or deducing patterns coupled with the quality of inputs relating to the outcomes of those inputs combined. All of these prior approaches suffer from deficiencies and lack the scalability to achieve results across institutions and beyond the typical “silo” solutions accordingly offered.

### SUMMARY OF THE INVENTION

The following summary is exemplary and is not necessarily restrictive of the invention as claimed.

Embodiments of the disclosed system are designed to manage information in clearly defined workflows to permit detailed analysis, intervention, training, metric measurements, problem solving frameworks, machine learning, real time updates, statistical process control, data base management, prediction and optimization.

Methods of the present invention include sequences of steps defining workflow and detecting structures in the data for event analysis and predictor functions comprising human factors, human reliability analysis, facility reliability analysis, optimal and suboptimal component interactions. By structuring and linking the components into optimal subsets an expert system is capable of detecting potential threats to quality, safety and outcomes.

Embodiments of the system are designed to match adherence to successful key drivers, key roles and training (correct completion of required protocols/tasks) to and/or with outcomes. The method structures data on observed skills from top outcome enterprises that are effectively applied to produce an optimal outcome in a given workflow. Hidden factors, human factors, facility resources and component interactions are linked to outcomes. Assessments are in the form of simulations, traditional testing, peer and management reviews. Embodiments of the present invention connect an organization, and instantly provide meaningful use of the data collected by immediately alerting the individuals who need to know whether certain activity is running on time, performed correctly and whether the protocol documentation is complied with. Embodiments of the present invention provide for features of a process to solve the continued issue that scattered, uncoordinated human observation in many industries cannot, through purely human means, provide for networked analysis and near-real-time problem identification and solution mapping.

The present invention overcomes the limitations and deficiencies in current performance based training systems, general systems and methods for performing an action on structured data, and efforts to identify factors contributing to outcome improvements or error detection in safety and quality among other workflows by combining and enhancing these approaches with redundancy in classification and analytics of data for an applied purpose to improve safety, quality and overall outcomes.

Embodiments of the present invention accelerate the transfer of knowledge from top-performing enterprises so that other similar enterprises are able to emulate the top performing enterprise's success for optimal outcomes in any one particular product or service line. This platform can be applied to any number of industries, product or service lines.

There is disclosed herein a platform comprised of a system(s) and method(s) to improve outcomes in any number of industries. One example of an industry where this platform can be applied is healthcare, especially hospital improvements in patient outcomes. Within the modalities of medicine and workflows within a hospital setting a potential application allows an institution such as a hospital to emulate success that some hospitals have achieved in reducing or nearly eliminating hospital acquired infections.

There are other industries, such as construction, that have many different roles and protocols along the work flow continuum, and features of the present invention relate to identification of key roles, by automatically, and unprompted by human intervention, communicating in real time on pertinent protocols, and simultaneously reaching the entire work flow continuum. The present disclosure relates to any work flow continuum that relies on timely communication, is interdependent on protocol compliance, is currently monitored and communication is by human observation alone without any automation of protocol compliance and or communication of relevant completion of protocols.

One preferred embodiment includes a method comprising: (a) identifying a tracked outcome to be monitored (e.g. reduce hospital required infections below a particular level); (b) correlating benchmarking data to tracked outcome (identify institution with acceptably low values and/or predetermined range to be satisfied); (c) assigning, based on the benchmarking data, a predetermined in-range and out-of-range threshold to the tracked outcome based on the benchmarking data; (d) obtaining designated events that affect the tracked outcome within the in-range and out-of-range thresholds; (e) monitoring designated events corresponding to the tracked outcomes for conformance to the predetermined in-range threshold for the corresponding tracked outcomes; and (f) determining that an out-of-range condition exists for the tracked outcome, and upon determining an out-of-range condition exists, determining a remediation process for mitigating the out-of-range condition by determining which designated event was a cause of the out-of-range tracked outcome. Further, in various embodiments, the steps (c)-(f) may be updated on a real-time or near-real-time basis. Additionally, determining the remediation process may include several optional steps and may further include assignment of a turnaround team to implement the remediation approach, or identifying a source of error leading to out-of-range condition, or identifying training to be provided to mitigate the source of error, or identifying personnel requiring training to mitigate the source of error. In various embodiments, continued process improvement may prevent systemic issues from arising, such as by identifying personnel and providing continual training (to staff, administration, or other personnel) as a prophylactic approach to reduce out-of-range conditions, or analysis of collected operational data to identify errors contributing to the out-of-range condition for the tracked outcome.

In various embodiments, reporting out of range conditions provides enhanced ROI and helps to prevent systemic or individual issues from causing deviation from desired outcomes. Thus reporting aspects of the present invention provide for either on-demand access or push-basis alerts, and provides for formatting a report for reporting in-range and out-of-range events corresponding to the tracked outcomes, or formatting an alert to institutional administration staff or patient care staff to be transmitted upon detecting that an out-of-range condition exists. Various embodiments provide for formatting a report showing real-time status of compliance with tracked outcomes, comparing in-range and out-of-range conditions over time. Further, as operational performance is tracked in real time or near-real time, the real-time status in various embodiments is presented as a virtual dashboard interface.

Embodiments of the present invention, as discussed more completely below, provide for cloud-based implementations, and any data associated with the present invention may be stored in networked or cloud-based databases. For example, collected operational data, benchmarking data, tracked outcomes, predetermined in-range and out-of-range conditions, reports, and any other data that is utilized in concert with the present invention may be stored in a cloud-accessible database, or in a local server, satellite server, or other conventional hosting mechanism.

Yet another embodiment provides for a computer-based system for detecting workflow, facilities and or resources anomalies leading to suboptimal outcomes in enterprises and performing action to detect potential optimal outcomes, safety and quality optimization, comprising: an input device for receiving data; an output device for presenting data; and a memory storing mechanism including methods comprising: a database; an analyzer; a user interface; and an action processor. And yet another aspect of the present invention provides a computer-based method consisting of a sequence of steps defining workflow components which comprise optimal outcomes in a given enterprise comprising: defining optimal inputs and their standards; detecting structure in the data; determining correlation among inputs; detecting safety breaches that could lead to suboptimal outcomes or endangering quality and safety conditions; matching or linking anomalies to structures in the data leading supporting optimal outcomes; and application of continuous moving algorithms.

Example of One Application of this Platform

An Addressable Problem

Hospitals administer their services in what amounts to an assembly line of continuum of care. Though the continuum of care is the result of a collective set of resources and talent the management and development of these resources and talented is usually conducted with a silo′d approach—separate and apart from each other. There exists no mechanism today to enable a hospital to infiltrate each silo to simultaneously manage change or improvements with training and metrics.

A hospital focus utility of this platform is designed to bridge medical expertise around the world enabling every hospital to learn and emulate how top patient outcomes can be achieved in a comprehensive and inter-professional manner, making it possible for the entire hospital to be trained and measured on improving patient outcomes.

The current modalities to transfer medical expertise are extremely costly, fragmented and not linked to outcomes. By combining a proprietary approach and leveraging new and existing forms of technology we are able to provide global access with a turnkey infrastructure for fractions of the cost for an entire hospital to improve patient outcomes. In this way, clinical successes can be “democratized,” enabling the world's top medical expertise to reach its highest potential in a scalable and cost-effective manner.

Key addressable market drivers include increased unreimbursed hospital expenses, government and market demands for better patient outcomes, and patients seeking alternatives to hospitals due to the risk of infection.

A pioneering a System as a Service “SaaS” platform to improve patient outcomes and cost containment in hospitals that enables the entire hospital to improve collectively—at the same time and on the same topics—inter-professionally and multidisciplinary is innovative, timely, and possible through implementations of the present invention.

With a turnkey platform that supports a continuously moving algorithm with real time benchmarking, dashboard, reporting, and an inter-professional training platform, this approach is designed to enable an accelerated transfer of knowledge and an ability for material improvements in patient outcomes.

Hospitals in the United States have begun to achieve and sustain zero or near zero rates of hospital acquired infections/incidents (HAIs) in the areas of catheter urinary tract infection (“CAUTI”), central line associated blood stream infection (“CLASBI”), patient falls, and ventilator associated pneumonia (“VAP”). This platform is designed to bridge throughout the world the medical expertise enabling hospitals to emulate zero/near zero HAIs. To this end, embodiments of the platform described herein provide for a SaaS cloud-based application with training, metrics and analytics built from evidence-based clinical research and interviews with the hospitals that have achieved the HAI turnaround.

Indications based on the success in the United States support the conclusion that a hospital can reduce their CAUTI infection rates by 10-50% in 6-12 months if able to emulate the success of other hospitals; this is possible through embodiments of the invention that provide a framework within which to operate that enable the human resource to improve in the areas that other hospitals with low infections rates have done.

Referring to the diagrams provided herein, and as discussed in more detail below, three interface points support clinicians all on handheld devices that either upload directly to the cloud or near field communication devices. The first interface is intended to collect input protocol checklists (insertion, process and maintenance) and patient profile information (a predetermined set of potential threats to infection that the clinician selects from). The second is a daily update delivered to the clinical care team indicating how many patients they are responsible for that have an indwelling urinary catheter, which patients contracted an infection or are symptomatic and timely removal alerts. The third is a weekly, monthly, quarterly summary report with trends and problem solving tools.

Initially the platform will be event driven analysis with additional features already in development such as alerting the infection control team of the need for an audit due to safety breaches detected along the continuum of care.

Currently no one is providing an inter-professional platform of this kind. The World Health Organization has recognized that inter-professional training leads to better conditions and employment relations among all professionals in the medical team; by improving the way the team works together, within the clinical team aligning with the administration, hospitals may capture all their strengths and perform better for the patient. However, inter-professional training is only an initial step in providing for a robust teamwork environment that functions through provision of information simultaneously disseminated throughout the inter-professional team, thus accelerating and providing additional integrated cross-team integration beyond the World Health Organization's recommendations.

Embodiments of the invention provide for ready-made infrastructure to train an entire medical team throughout the continuum of care within a hospital. In various platform embodiments, the medical team can align clinical care with cost containment objectives through the continuous moving algorithm and real time benchmarking.

A Hospital Application of this Platform

In one embodiment, a hospital application of this platform is organized by subspecialty areas of medicine. Hospitals will be able to license subspecialty training for all clinicians along the continuum of care on a per-site time limited basis. One initial focus, for example, may be directed toward eliminating Hospital Acquired Infections, currently a $33 billion annual non-reimbursed expense in the US accounting for 75,000 deaths and infecting over 700,000 people a year (source Center for Disease Control).

Competitive Advantage for Participating Hospitals

Embodiments of the present invention align the business side with the clinical care and training efforts to improve patient outcomes through our continuous moving algorithm and real time benchmarking. A hospital can then better focus on reducing issues such as Hospital Acquired Infections together supporting the clinical turnaround and aligning it with cost containment.

### BRIEF DESCRIPTION OF THE DRAWINGS

A more complete understanding of the present invention may be derived by referring to the detailed description and claims when considered in connection with the following illustrative figures.

FIG. 1 illustrates a block diagram of an exemplary system and method of the present invention.

FIG. 2 shows a linked block diagram with the salient features of this platform.

FIGS. 3 and 4 illustrate block diagrams that together demonstrate different approaches into an enterprise which in this example is a hospital.

FIG. 5 provides a diagram illustrating resources that require active know-how in order to achieve the desired goals and outcomes.

FIG. 6 illustrates the combination of workflow structures combining with analytical tools to provide useful uses of data that enable a team to learn how best to optimize their work efforts.

FIG. 7 illustrates an exemplary a block diagram of the platform's structure.

FIG. 8 illustrates development components that interact to deliver the one platform embodiment of the present invention.

FIG. 9 shows a block diagram depicting user interfaces of the present invention.

### DETAILED DESCRIPTION

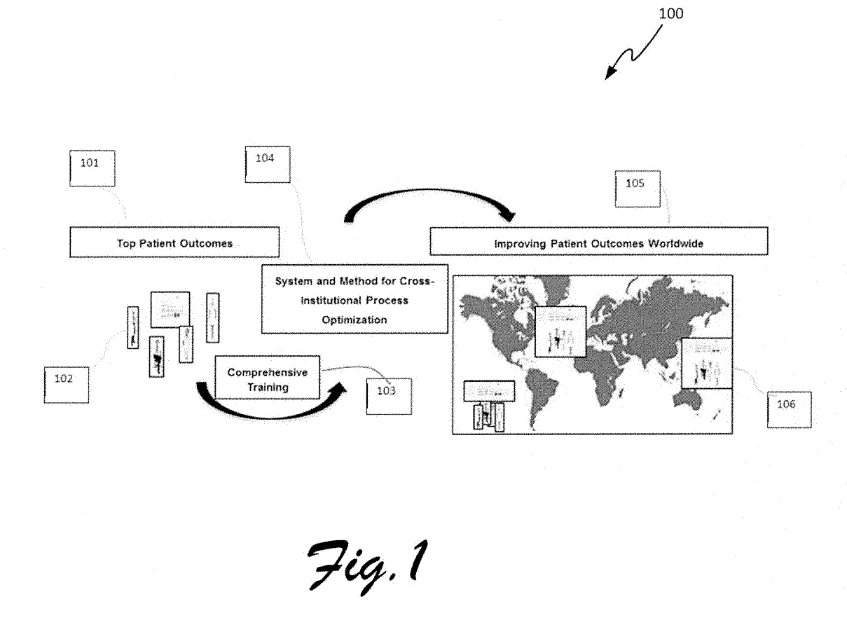

Referring now to FIG. 1, there is provided a high-level block diagram 100 of an embodiment of the present invention showing data flowing from one enterprise's top outcomes 101 to another, starting by identifying a top outcomes enterprise (that is, identifying an entity such as a particular hospital or institution that meets a predetermined outcome criterion, such as acquired infection rate). FIG. 1 also shows an illustration of a hospital with top outcomes, comprised of a multidisciplinary approach which is then decomposed for methods of training and assessment to then be linked to other data which quantifies workflow's improved value add then distributes it as a based platform to other hospitals focused on the same modalities of medicine or attempting to solve the same deficiencies in outcomes. Special attention is given to selecting the area in which the enterprise performs at a top outcomes level focusing on critical systems and outcomes. Once the top outcomes enterprise is identified and the specific outcome feature determined such as a hospital with no or hospital acquired infections or a nuclear power plant with no accidents on record, then the entity (such as a hospital system) enabling that outcome is focused on as depicted in 102. This system comprises individual methods embodied in different roles throughout the organization with the entire enterprise taken into account for a systemic approach. Training 103 is linked to factors contributing to outcomes emulating both the top outcomes enterprise as well as other sources demonstrated to enhance outcomes or that can be tested to determine the effect of such training on outcomes. The platform depicted in 104 engages with the factors described above in 101 through 103 with a series of methods and systems designed according to data classifications and goals to deliver a scalable solution for other enterprises. Embodiments provide a scalable financially viable platform 105 where the reach is global and can be delivered with sensitivity to cultural approaches enabling enterprises in the same or similar industries to share workflow optimization and improve outcomes. Enterprises in the same or similar industries produce similar deliverables and depend on systems and methods to reach standards in quality and safety that would uphold top outcomes. The provided platform has the ability to scale to multiple enterprises, industries and sectors simultaneously delivering the ability to emulate and evolve capacities for an enterprise to improve outcomes.

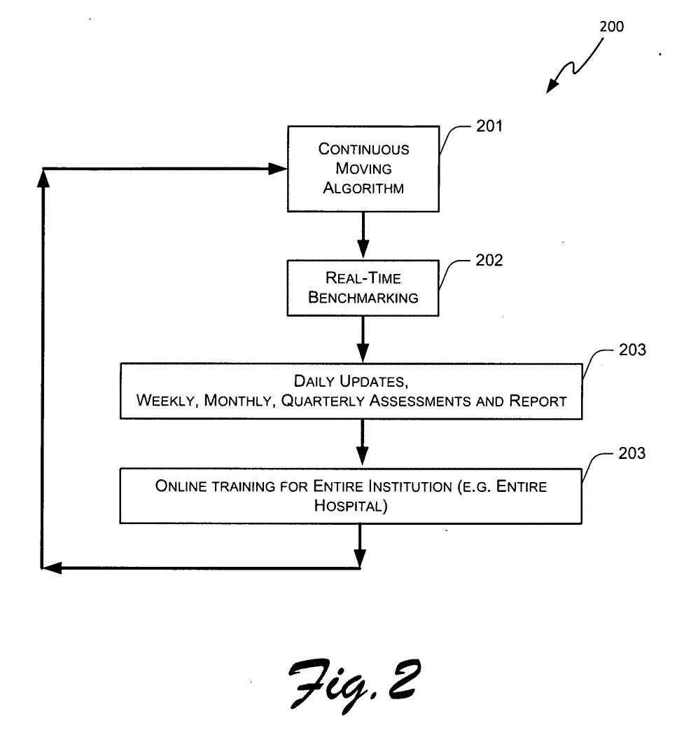

FIG. 2 shows a block diagram with the salient features of this platform, including a continual process for cross-institutional improvement. By applying the methods of classification and analytics into one platform a continuous moving algorithm combines with real time benchmarking to provide snapshots and reports on progress, safety breaches, trends, and training. In FIG. 2 the qualities of the platform whose underpinnings are supported by the methods and systems in the platform is described. The platform contains the ability to perform a continuous moving algorithm 201, algorithms are developed to motivate the methods and support the system's operative functions. The algorithms function with real-time data and therefore are continuous in their calculations. The platform classifies data and conducts calculations 202 within real time dynamics delivering the opportunity for user interfaces on a real time benchmarking tracking progress and identifying opportunities for real time improvement and ability to amend potential safety breaches just in time of escalated errors developing. In one example, the tracking algorithm monitors events (such as reported instances of hospital required infections) and as the events exceed a particular criterion (such as a minimum threshold showing infections have begun to increase beyond a desired level) then factors correlated with the event change are identified and reported. User interfaces 203 which communicate with a variety of roles within an enterprise according each role's responsibility as it contributes to outcomes, and thus the accumulation of each input to the process may be monitored and correlated with monitored outcomes. In support of the improvements is 24/7 training 204 accessible to the entire enterprise according to the skills and knowledge each role requires the individual and contribute to top outcomes, and such training opportunities may be identified as part of the real time benchmarking and monitoring (such as proper sanitary procedure being implemented for new patient admission). The individual steps may operate continuously, in real time, and are updated as the arrows indicate to support a process of continued improvement.

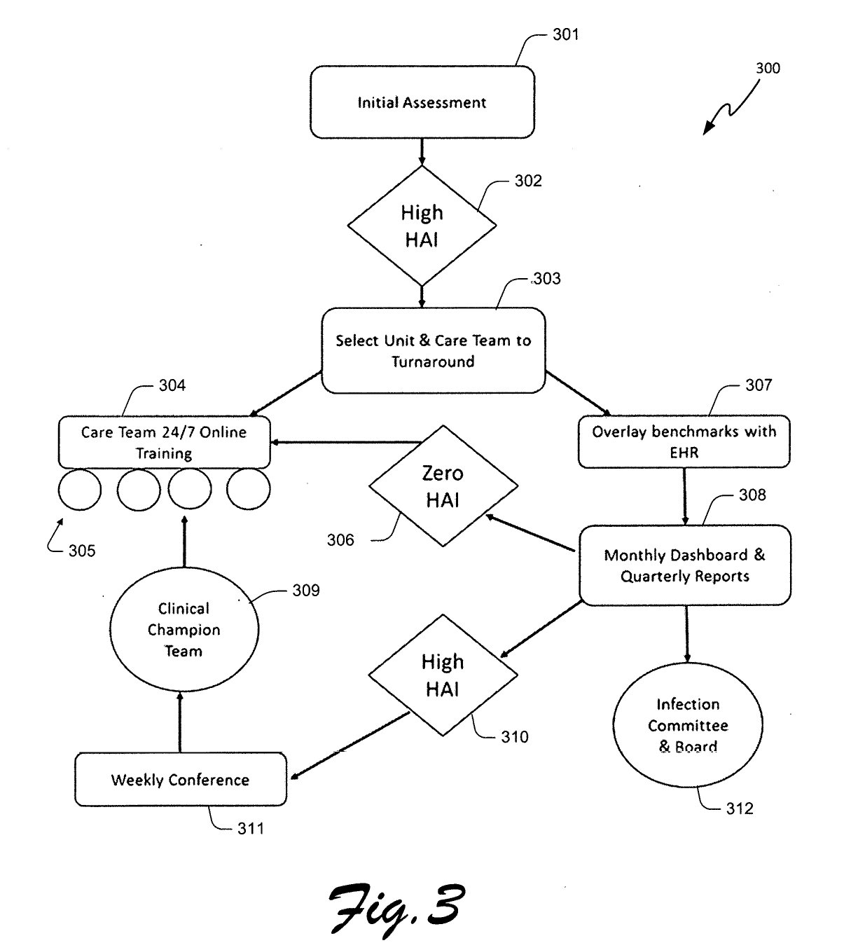

FIG. 3 depicts an example 300 of an application of this platform's system in a hospital setting, the approach is similar for other industries. An initial assessment 301 the platform form utilizes a series of methods such as interviews, surveys, and work observation to determine factors that contribute to the outcomes and assesses the enterprises to be improved, along with current ability to deliver the top tier outcomes in a given area or focus of medicine. Once a high error level 302 (or monitored events for outcomes going beyond a predetermined acceptable range) is determined, the data collected is classified to identify errors contributing to the out-of-range outcome. For example, an out of range condition may be that a level of hospital acquired infections has increased beyond a predetermined threshold, for example 0.5%. Based on certain attributes a turnaround team 303 is selected at the hospital; this team is selected to champion, motivate and manage the hospital's adherence to the new platform and its engagement and dedication towards improving the patient outcomes. In support of the improvements is 24/7 training 304 accessible to the entire enterprise according to the skills and knowledge each role requires the individual and contribute to top outcomes. Individual team members 305 receive tailored training to support them in their role to optimize outcomes. Individuals participation in this training is then matched to that individual's patient outcomes for the clinical care teams they are on, in effect creating a performance based training platform that is correlated in real time to outcomes enabling a hospital to perform return on investment (ROI) analysis on the training and identify areas of improvement overall in the system, processes and individual capabilities. If data classification does not identify any safety breaches or infections 306 then the training is continued. Optimal benchmarks from enterprises are overlaid 307 with in-house data in the hospitals under improvement to determine the gaps and differences between the two enterprises in achieving top outcomes. A user interface with dashboards and reports enables 308 users in both the operation of the enterprise or management; in the hospitals it would be clinical care team, support staff, administration, board and c-suite as well as other levels of management, to access data classifications in user friendly formats and in different time sequences. The team selected for the turnaround 309 collaborates with their top tier peers to learn how to optimize and resolve concerns. If data classification signals errors 310 or in the case of hospitals infection rates above the top tier outcome levels, then periodic in person coaching, mentoring, or general contact with the top tier hospital is implemented to enable cross institutional in 311 person discussion. User interface 312 with executives, managers, administrators, board members and all or any others with oversight responsibility convene in a committee or meeting setting to share insights and review progress or ongoing obstacles providing transparency to the process.

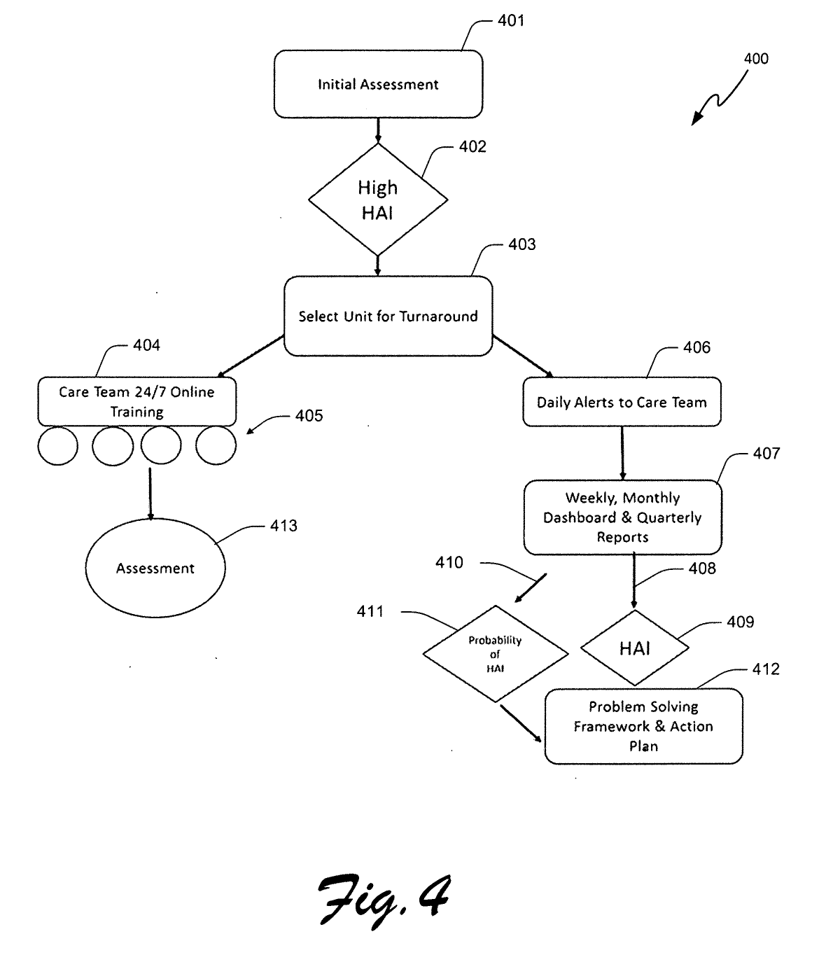

In FIG. 4, there is provided as an example of an application of this platform's system 400 in a hospital setting, and embodiments are similar for other industries. An initial assessment 401 the platform form utilizes a series of methods such as interviews, surveys, and work observation to determine factors that contribute to the outcomes and assesses the enterprises to be improved's current ability to deliver the top tier outcomes in a given area or focus of medicine. Once a high error level 402 or in this case a level of hospital acquired infections that can be improved upon given other hospital's top outcomes, the data collected is classified to identify errors contributing to the low outcome. Based on certain attributes a turnaround team 403 is selected at the hospital this team is selected to champion, motivate and manage the hospital's adherence to the new platform and its engagement and dedication towards improving the patient outcomes. In support of the improvements is 24/7 training 404 accessible to the entire enterprise according to the skills and knowledge each role requires the individual and contribute to top outcomes. Individual team members 405 receive tailored training to support them in their role to optimize outcomes. Individuals participation in this training is then matched to that individual's patient outcomes for the clinical care teams they are on, in effect creating a performance based training platform that is correlated in real time to outcomes enabling a hospital to perform return on investment (ROI) analysis on the training and identify areas of improvement overall in the system, processes and individual capabilities. 406 User interface possible with the continuous data classification enables visibility between real time operators and outcomes. User interface with dashboards and reports enables 407 users in both the operation of the enterprise or management, in the hospitals it would be clinical care team, support staff, administration, board and c-suite as well as other levels of management, to access data classifications in user friendly formats and in different time sequences. 408 if safety breaches or errors are detected 409 the data is matched to outcome probabilities and places an alert signaling an infection or potential for an infection. 410 if no error or infection is detected then a predictor 411 of any safety breaches that are material to infections are looked for. 412 user interface with the system to structure the data classified and records the team's decision on how to handle the errors leading to poor outcomes. These adjustments are added to the system as inputs and tracked for their effectiveness. 413 assessments of both individual knowledge and skill as well as assess the effect this training is having on outcomes allowing for ROI analysis on training, personnel development, process and policy effectiveness.

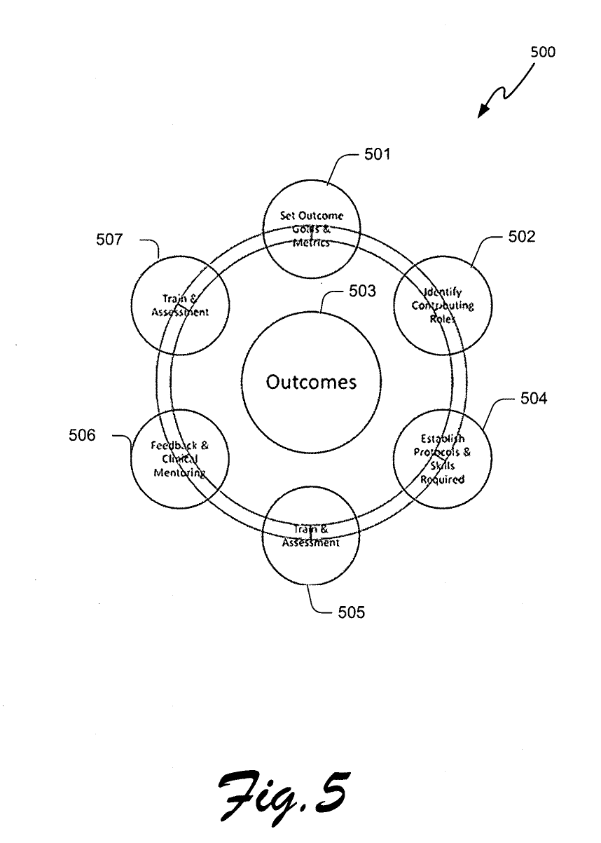

FIG. 5 outcomes are supported by a number of systems, and improving defined outcomes 503 is an aim of embodiments of this invention. In this figure the description for a few of the salient features this invention enables an enterprise to perform 501 to achieve desired enterprise objectives, goals for quality of outcomes are established and matched with metrics. Numeral 502 shows the identification of roles that operate along the continuum or process enables training and metrics to be formed to assess progress and track inputs affecting desired outcomes along the way. Thus, the activities shown the diagram 500 are focused on tracking and improving the quality of the outcomes. Top tier outcomes 504 are achieved by finely tuned protocols and skills that directly affect outcomes. These factors are determined through a number of methods some of which include interviews, surveys, observed behavior. These factors are inputs in our platform and are then classified through our systems and methods to determine optimal inputs, variants, significance, thresholds, measurable junctions and other salient features to optimal outcomes. Selection of training and assessments 505 specifically contribute to outcomes as described above. Feedback and mentoring 506 support specific skill sets developed and nurtured in the training and assessment phase and applied in managing processes that contribute to outcomes. As a result, the present invention provides a mechanism and structure to develop optimal feedback and mentoring skill sets based on top tier outcomes achieved within the industry. Continuous improvement is also provided by recalibrating 507 the training and assessment content, methods, and intention, and in various embodiments, this can be accomplished effectively through our platform's ability to classify data.

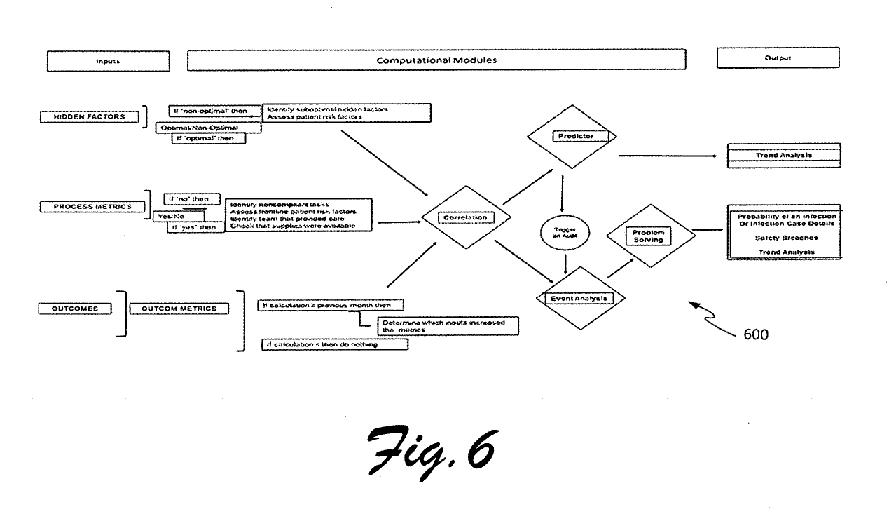

FIG. 6 features an overview of the systems and methods, workflow and components of one embodiment of a platform of the present invention. At the top, inputs collected from observable actions are taken by individuals that contribute to outcomes. In this example it is in the healthcare setting focusing on an infection acquired in the hospital also known as nosocomial infections. Hidden factors are common in critical systems and are deduced scientifically, mathematically and with applied psychology and industrial engineering methods, all of which are present in our model through modules within the system and the methods of collecting information. Process metrics are the inputs that are protocol driven and pertain to clinical and administrative methods of practicing medicine or managing a hospital setting. Outcomes are the measurable results though not necessarily patient outcomes pertaining to the patient's well-being, these metrics pertain to outcomes of policy and protocols. Computational modeling is the platform itself and is comprised of systems and methods control flow statements, statistical process controls, event analysis, correlations, predictors, and databases among others. A control flow statement is related to the hidden factors, another control flow statement is related to the protocols. A is the metric calculations from the metric inputs referred to above. There is also provided an example of the deductive reasoning identifying the contributing factors that led to the outcome of the control flow statements for hidden factors, and further, an example is provided of the deductive reasoning identifying the contributing factors that led to the outcome of the control flow statements for protocols. Further, an example of the deductive reasoning identifying the contributing factors that led to the outcome of the control flow statements for outcomes referred to above is shown. Further, storage and classification of the contributing factors referenced in the control flow statements that are correlated as above are sorted as either an infection event or non-infection event both of which refer to our ultimate goal of emulating top tier infection levels at their lowest achievable level. Additionally, data is further classified to determine predictor levels of possible infections based on safety breaches and other classifications of the data. Any safety breaches detected trigger an audit, even in the specific unit and with the specific personnel identified in the safety breach and other classifications that indicate a possible infection threat of above a defined parameter. If an infection rate is detected in the correlation module, an event analysis is initiated. Once the safety breach is audited an event analysis is conducted. Following the event analysis, data is reclassified and matched with solutions within a framework to narrow down salient data reflecting behavior and this classification of data is labelled, sorted and communicated that can be addressed by management. Summaries of probability of an infection event, the case specifics of an event, safety breaches and trend analysis are a few examples of a report that is issued following the problem solving module. This falls under the output sequence which engages user interfaces. In the scenario where no infection and no safety breaches or any other negatively correlated feature to optimal outcomes is detected then a trend analysis is produced along with other user interface features all designed to enable successes to be identified and replicated within the enterprise.

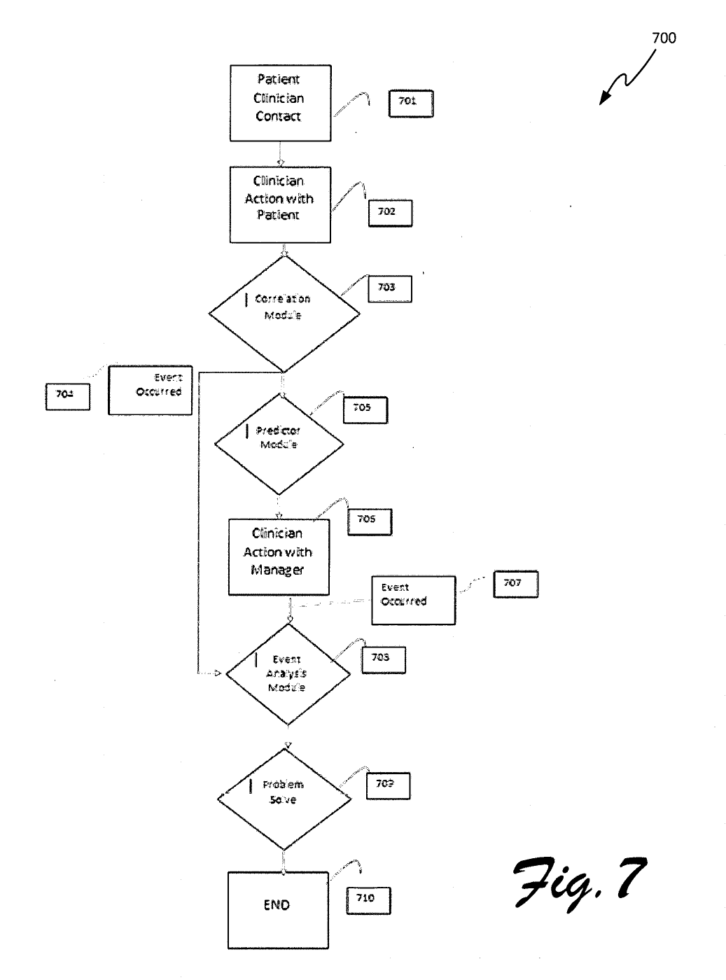

FIG. 7 depicts one embodiment of start to end process of the platform described in FIG. 6 in summary form, and provides a block diagram of one embodiment of the platform's structure. Key inputs are identified, correlated and signaled as on track or not on track to meet the desired outcome. The on-track signal then processes the information and draws on learned data to determine if a safety threat to the process exists. All data is then analyzed and deductions are made to teach appropriate next right actions to meet desired goals and outcomes.

Patient care 701 consists of several different contact points between clinicians, support staff and patient. These activities each contribute to outcomes and are a starting point of the exemplary platform. Each clinician's action 702 is governed by distinct activity which is captured and recorded in a database of the present system (not shown), and a multitude of clinician contact points with each other, the patient, administration and machine or devices or medications are captured and recorded 703 as per these factor's correlation to the outcome. In the data classification process 704 if an error or safety breach occurs, the system moves to a method for event analysis 708. If no error or safety breach event is detected in step 703, then a predictor method and system is applied 705 to the classified data. In such a case, clinician action with manager is undertaken 706 by alert provided by a report to review the event, non-event and user interface to determine course of action and event analysis is performed 703. If an event has occurred 707 that is not an infection but an indication of significant prediction of an infection given the statistical control process and methods' signally such a prediction, an immediate event analysis 708 is conducted. Further, a problem solving techniques are analyzed and recommended 507 to embody a specific set of skills, methods, systems and deduction to discern cause and effect leading to an error or infection event or safety breach. The process ends 710 once the problem solving module has classified data.

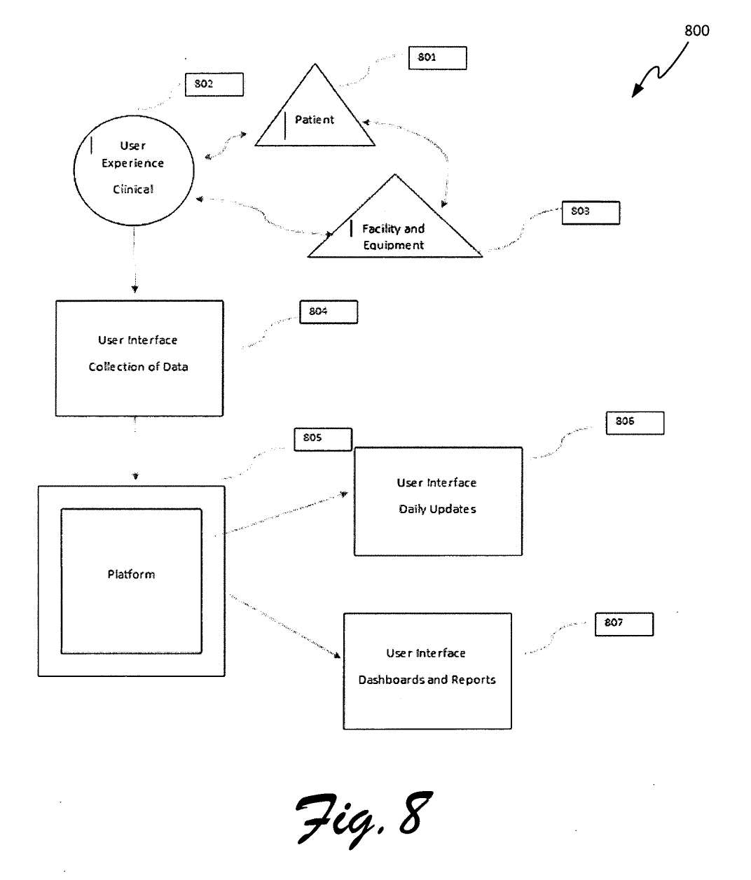

FIG. 8 illustrates another view 800 of the interaction of component in summary form. Step 801 the starting point is the patient. At step 802, the clinician engages with the platform and the patient and the facility. Further, at step 803 the facility represents a number of components including the physical environment, enterprise culture, device and equipment among others. A user interface to collects data 710 to form an important component and is to be conducted in a number of modalities including hand held devise, computer, paper, interviews, audits and user led initiatives that may not be traditional implemented designs. Also, the platform 805 comprises receiving data, storing data, classification of data, predictors, event analysis, human reliability analysis, component interaction analysis, clustering, machine learning, statistical process control, linear regression, and other statistical, computer science, industrial engineering, psychology and medical or industry specific disciplines in respective databases (not shown). A User interface 806 makes continuous data classification possible and enables visibility between real time operators and outcomes. A second user interface 807 provides dashboards and reports that enables users in both the operation of the enterprise or management, in the hospitals it would be clinical care team, support staff, administration, board and c-suite as well as other levels of management, to access data classifications in user friendly formats and in different time sequences.

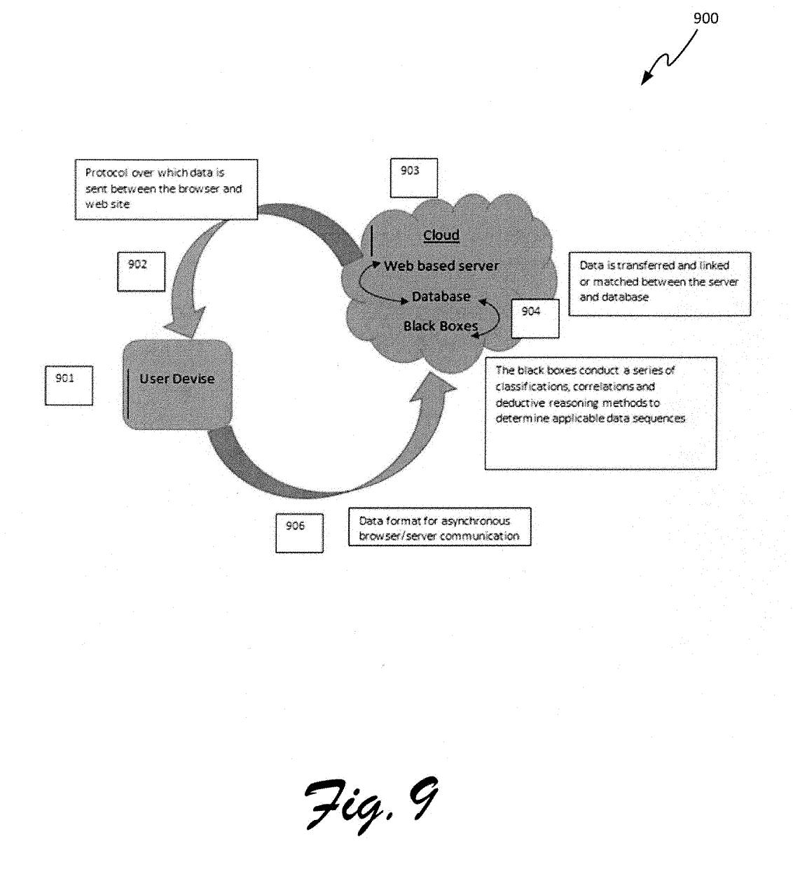

FIG. 9 is an illustration of the user device and cloud, and those of skill in the art appreciate that cloud computing 903 is a kind of Internet-based computing that provides shared processing resources and data to computers and other devices on demand. A user device 901 will be utilized to collect data as well as to deliver information in the form of daily updates, dashboards, and reports. An interface 902 provides a protocol over which data is sent between, for instance, a browser and web site. As shown within the cloud, data is transferred and linked or matched 904 between the server and database. Modules for classification and analysis of data may then be used to conduct a series of correlations and deductive reasoning methods to determine applicable data sequences to improving outcomes. Further, a protocol is shown for data format 906 for asynchronous browser server communication and user devise.

The particular implementations shown and described above are illustrative of the invention and its best mode and are not intended to otherwise limit the scope of the present invention in any way. Indeed, for the sake of brevity, conventional data storage, data transmission, databases, and other functional aspects of the systems may not be described in detail. Methods illustrated in the various figures may include more, fewer, or other steps. Additionally, steps may be performed in any suitable order without departing from the scope of the invention. Furthermore, the connecting lines shown in the various figures are intended to represent exemplary functional relationships and/or physical couplings between the various elements. Many alternative or additional functional relationships or physical connections may be present in a practical system.

Changes and modifications may be made to the disclosed embodiments without departing from the scope of the present invention. These and other changes or modifications are intended to be included within the scope of the present invention, as described in this disclosure, and as expressed in the following exemplary claims.

What is claimed is:

1. A method comprising:
(a) identifying a tracked outcome to be monitored (e.g. reduce hospital required infections below a particular level);
(b) correlating benchmarking data to tracked outcome (identify institution with acceptably low values and/or predetermined range to be satisfied);
(c) assigning, based on the benchmarking data, a predetermined in-range and out-of-range threshold to the tracked outcome based on the benchmarking data;
(d) obtaining designated events that affect the tracked outcome within the in-range and out-of-range thresholds;
(e) monitoring designated events corresponding to the tracked outcomes for conformance to the predetermined in-range threshold for the corresponding tracked outcomes; and
(f) determining that an out-of-range condition exists for the tracked outcome, and upon determining an out-of-range condition exists, determining a remediation process for mitigating the out-of-range condition by determining which designated event was a cause of the out-of-range tracked outcome.

2. The method of claim 1, wherein steps (c)-(f) are updated on a real-time or near-real-time basis.

3. The method of claim 1, wherein determining the remediation process further includes assignment of a turnaround team to implement the remediation approach.

4. The method of claim 1, wherein determining the remediation process further includes identifying a source of error leading to out-of-range condition.

5. The method of claim 1, wherein determining the remediation process further includes identifying training to be provided to mitigate the source of error.

6. The method of claim 1, wherein determining the remediation process further includes identifying personnel requiring training to mitigate the source of error.

7. The method of claim 1, further comprising providing continual training as a prophylactic approach to reduce out-of-range conditions.

8. The method of claim 1, further comprising identifying personnel to receive continual training as a prophylactic approach to reduce out-of-range conditions.

9. The method of claim 1, further comprising: formatting a report for reporting in-range and out-of-range events corresponding to the tracked outcomes.

10. The method of claim 1, further comprising: formatting a report for reporting in-range and out-of-range events corresponding to the tracked outcomes.

11. The method of claim 1, wherein the determining a remediation process further comprises analysis of collected operational data to identify errors contributing to the out-of-range condition for the tracked outcome.

12. The method of claim 11, wherein the collected operational data is stored in a cloud-accessible database.

13. The method of claim 1, further comprising: formatting an alert to institutional administration staff to be transmitted upon detecting the out-of-range condition exists.

14. The method of claim 1, further comprising: formatting an alert to patient care staff to be transmitted upon detecting the out-of-range condition exists.

15. The method of claim 1, further comprising: formatting a report showing real-time status of compliance with tracked outcomes, comparing in-range and out-of-range conditions over time.

16. The method of claim 15, wherein the real-time status is presented as a virtual dashboard interface.

17. The method of claim 1, wherein tracked outcomes are stored in a cloud-accessible database.

18. The method of claim 1, wherein benchmark data is stored in a cloud-accessible database.

19. A computer-based system for detecting workflow, facilities and or resources anomalies leading to suboptimal outcomes in enterprises and performing action to detect potential optimal outcomes, safety and quality optimization, comprising:
an input device for receiving data;
an output device for presenting data; and
a memory storing mechanism including methods comprising:
a database;
an analyzer;
a user interface; and
an action processor.

20. A computer-based method consisting of a sequence of steps defining workflow components which comprise optimal outcomes in a given enterprise comprising:
defining optimal inputs and their standards;
detecting structure in the data;
determining correlation among inputs;
detecting safety breaches that could lead to suboptimal outcomes or endangering quality and safety conditions;
matching or linking anomalies to structures in the data leading supporting optimal outcomes; and
application of continuous moving algorithms.
